(19) United States
(12) Patent Application Publication
(10) Pub. No.: US 2008/0172071 A1
(43) Pub. Date: Jul. 17, 2008

(54) Umbilical or Paraumbilical Hernia Repair Prosthesis

(76) Inventor: Stephen George Edward Barker, London (GB)

Correspondence Address: HARNESS, DICKEY & PIERCE, P.L.C., P.O. BOX 828, BLOOMFIELD HILLS, MI, 48303, US

(21) Appl. No.: 11/632,822

(22) PCT Filed: Jul. 20, 2004
(86) PCT No.: PCT/GB04/03144

§ 371 Date: Dec. 19, 2007

(51) Int. Cl. A61F 2/02

(52) U.S. Cl. 606/151

(57) ABSTRACT

An implantable prosthesis for the repair of muscle wall defects such as hernias and more particularly for the repair of umbilical and paraumbilical herniae and possibly ventral or Spigelean herniae, comprises an upper sheet (1) of a surgically compatible mesh or PTFE material and a lower button (2) of a surgically compatible mesh or PTFE material (3), such as a composite polypropylene mesh/PTFE, connected with, but spaced from, the upper sheet by a bridge portion (5). The innermost surface of the button (2) includes a visceral adhesion inhibiting material (4) of a PTFE, or comparable material.

This invention relates to an implantable device concerning, principally a prosthesis for the repair of umbilical and paraumbilical herniae where the hernia bulge occurs at, or adjacent to, the umbilicus itself. The device may be used at other sites also, such as for a small ventral hernia, or a Spigelean hernia, for example. Such hernias are generally small, but can enlarge considerably. Previously, repair has been effected by means of a simple closure of various kinds, but such closures are “tensioned” repairs and can result in high recurrence rates. Flat, surgically acceptable mesh material has been used to close over the defect but often a distressing bulge remains and in some instances, the recurrence rate remains high. Repair beneath the muscle wall gives better results, but at the risk of internal adhesion formation to the prosthesis or significantly greater dissection.

Specialist prosthetic mesh materials have been suggested for umbilical/paraumbilical hernia repair and such mesh includes PTFE (polytetrafluoroethylene or equivalent material) as a coating on one side providing a “non-stick” characteristic. This material is suggested to be placed beneath the muscle layer of the abdominal wall or intra-abdominal, but involves a more extensive dissection than would normally be required. The technique is therefore, often avoided, with the coated mesh placed anterior to the muscle wall, over the defect.

It is a principal object of this invention to provide a prosthesis primarily for the repair of umbilical/paraumbilical herniae which has good load dissipating properties and which requires less dissection for insertion, utilising the benefits of a one side, non-stick component, with the alternate side designed to be incorporated into surrounding tissues.

According to this invention there is provided an implantable prosthesis for the repair of muscle wall defects such as occur in herniae, the prosthesis comprising an upper sheet of a surgically compatible material, for incorporation into surrounding tissues, and a lower button of a surgically compatible material connected with, but spaced from, the upper sheet, the innermost surface of the button including, or comprising, a visceral adhesion inhibiting material, such as PTFE.

The upper sheet and lower button may be connected through a centrally positioned bridge portion which may comprise a part conveniently formed by deformation of the button in the middle region for example. The sheet and button lie in generally parallel planes and in use embrace the muscle wall from opposed sides of the defect, connected by the bridge portion passing through the muscle wall defect. The sheet overlies the defect and surrounding healthy tissues and serves for sutured connection and reinforcement on the anterior aspect of the abdominal wall muscle layer, in a tension-free manner and may comprise mesh materials well known in the art for this purpose. The button may comprise a composite material such as a polypropylene mesh (to maximise growth of surrounding tissue adjacent the muscle) and with the opposed coating of visceral adhesion inhibiting material being of PTFE, or similar material.

The button will be smaller than the upper sheet to facilitate placement through the defect, which could be intra-abdominal, or pre-peritoneally placed. In a modification the button is collapsed or folded and may be opened after passage through the defect. The sheet, and button to some degree, may be cut according to requirements at the time of operation, and may be circular, rectangular or other shapes. The material of the button may be biodegradable.

An important feature of this invention is the two-part construction of the upper sheet member and the lower button of smaller dimension to facilitate passage through the defect.

An embodiment according to this invention is described and illustrated with reference to the drawings, wherein:

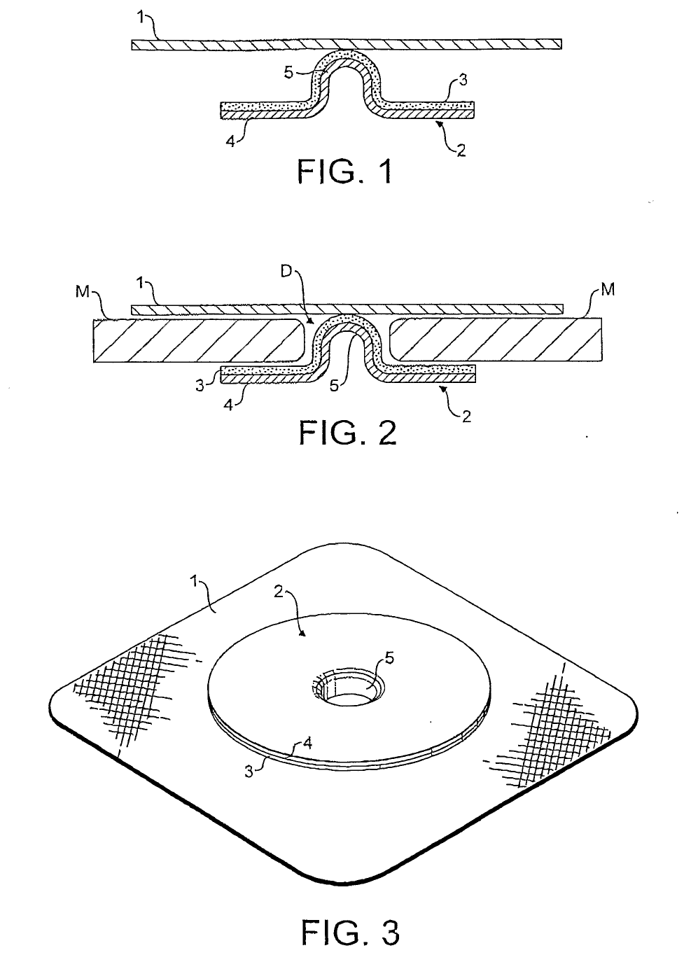

FIG. 1 is a transverse cross-section through a typical prosthesis according to this invention,

FIG. 2 shows the prosthesis of FIG. 1 in situ and diagrammatically,

FIG. 3 shows a perspective view of the prosthesis from the lower side,

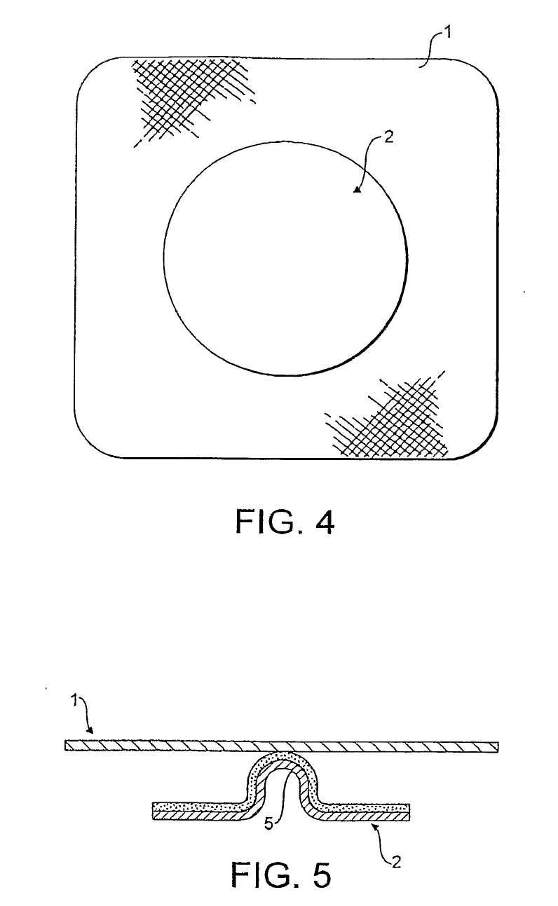

FIG. 4 shows in plan view the lower side and dimensions of one version, and

FIG. 5 show a side view as FIG. 4.

Referring to the drawings an implantable prosthesis is intended, primarily for the repair of muscle wall defects such as hernias and more particularly for the repair of umbilical and paraumbilical herniae and possibly ventral or Spigelean herniae, comprises an upper sheet 1 of a surgically compatible mesh or PTFE material and a lower button 2 of a surgically compatible mesh or PTFE material 3, such as a composite polypropylene mesh/PTFE, connected with, but spaced from, the upper sheet by a bridge portion 5. The innermost surface of the button 2 includes a visceral adhesion inhibiting material 4 of a PTFE, or comparable material.

The upper sheet 1 and lower button 2 are connected through the bridge 5 and this may comprise a separate connection part but here is shown more conveniently formed by deformation of the button into a middle protrusion. The sheet 1 and button 2 lie in generally parallel planes and in use embrace the (rectus) muscle wall M from opposed sides of the defect, with the button passing via the defect to lie adjacent to peritoneum/bowel, connected by the bridge portion 5 extending through the defect opening D (see FIG. 2).

The sheet 1 may comprise a standard polypropylene mesh material for suture to the surrounds of the region of the defect. The button 2 may be comprised of a material being a laminated polypropylene mesh 3 with a PTFE coating, or layer 4. The layer 4 may extend around the edges of the mesh 3. Other proprietary materials may be used subject to compatibility, for example all PTFE both textured and non-textured. The term “textured” being well understood by those versed in the art, is not exemplified further.

In an example, the sheet 1 was 70 mm square (typical range between 60 and 100 mm) with 10 mm radiused corners and the button 2 was 40 mm diameter, typical range 30 to 60 mm. The sheet and button were spaced by 8 mm, typical range 7 to 10 mm. A range of sizes, of similar dimensions, could be constructed.

In use the button is passed through the defect opening, necessitating less extensive dissection given the size, to come to rest adjacent to underlying peritoneum, or bowel (i.e. intra-abdominal) and the upper sheet is sutured in position to the anterior aspect of the abdominal wall muscle sheath, providing a tension-free repair and dissipating load. The material 1 and 3 aims to incorporate into the surrounding tissue. The PTFE coating 4 prevents the bowel, in particular, from adhering.

1-11. (canceled)

12. An implantable prosthesis for use in the repair of a muscle wall defect, the prosthesis comprising:
a sheet of a surgically compatible material, for incorporation into surrounding tissues;
a button of a surgically compatible material; and
a bridge portion of a surgically compatible material connecting the sheet to the button;
wherein prior to the placement of the button through the defect the sheet is spaced from the button by the bridge portion, and further wherein the surface of the button intended to face towards the viscera comprises a visceral adhesion inhibiting material and the button is smaller than the sheet to facilitate placement of the button through the defect.

13. An implantable prosthesis as claimed in claim 12, wherein the visceral adhesion inhibiting material is PTFE.

14. An implantable prosthesis as claimed in claim 12, wherein the button comprises a sheet of a surgically compatible material which is deformed in its middle region at least partially to form the bridge portion.

15. An implantable prosthesis as claimed in claim 12, wherein, prior to use of the prosthesis, the sheet and button lie in generally parallel planes.

16. An implantable prosthesis as claimed in claim 12, wherein the sheet and button are arranged, in use, to embrace a muscle wall from opposed sides of the defect, with the bridge portion passing through the defect.

17. An implantible prosthesis as claimed in claim 12, wherein the sheet is arranged, in use, to overlie the defect and surrounding healthy tissues and to serve for sutured connection and reinforcement on the anterior aspect of the abdominal muscle wall sheath layer, in a tension-free manner.

18. An implantible prosthesis as claimed in claim 12, wherein the sheet comprises a surgically compatible mesh material.

19. An implantable prosthesis as claimed in claim 12, wherein the material of the button is biodegradable.

20. An implantable prosthesis as claimed in claim 12, wherein the button comprises a composite material.

21. An implantable prosthesis as claimed in claim 20, wherein said composite material comprises a polypropylene mesh coated with the visceral adhesion inhibiting material.

22. An implantable prosthesis as claimed in claim 12, wherein the button is collapsible or foldable to enable the button to be collapsed or folded so as to pass it through the defect and to be opened out to be spaced from the sheet after its passage through the defect.

23. An implantable prosthesis as claimed in claim 12, wherein the sheet is capable of being cut.

24. An implantable prosthesis as claimed in claim 12, wherein the button is capable of being cut.

25. An implantable prosthesis as claimed in claim 12, wherein the sheet and/or button is circular or rectangular.

26. An implantable prosthesis as claimed in claim 12, wherein the sheet is generally square in shape, with the sides measuring between 60 mm and 100 mm in length.

27. An implantable prosthesis as claimed in claim 26, wherein the square sheet has radius corners.

28. An implantable prosthesis as claimed in claim 27, wherein the corners have a 10 mm radius.

29. An implantable prosthesis as claimed in claim 12, wherein the button is circular in shape.

30. An implantable prosthesis as claimed in claim 29, wherein the button has a diameter, range 30 to 60 mm.

31. An implantable prosthesis as claimed in claim 12, wherein the button is spaced from the sheet by an amount, range 7 to 10 mm.
